(19) United States
(12) Patent Application Publication
(10) Pub. No.: US 2008/0312330 A1
(43) Pub. Date: Dec. 18, 2008

(54) Methods for Reducing Mortality and Morbidity by Postoperative Administration of a Pharmacologic Cardiovascular Agent

(76) Inventor: Dennis T. Mangano, Hillsborough, CA (US)

Correspondence Address: SUTHERLAND ASBILL & BRENNAN LLP, 999 PEACHTREE STREET, N.E., ATLANTA, GA, 30309, US

(21) Appl. No.: 11/462,272

(22) Filed: Aug. 3, 2006

Related U.S. Application Data
(63) Continuation of application No. 09/426,792, filed on Oct. 22, 1999, now abandoned.
Application No. 09/426,792 is a continuation of application No. 08/787,056, filed on Dec. 3, 1996, now abandoned.

(51) Int. Cl. A61K 31/165; A61P 9/00

(52) U.S. Cl. 514/620

(57) ABSTRACT

The present invention relates to methods for reducing mortality and cardiovascular morbidity following surgery. In particular, the invention relates to the intensive postoperative administration of a pharmacologic cardiovascular agent to reduce mortality and cardiovascular complications. The invention is illustrated by way of working examples which demonstrate that in patients with, or at risk for, coronary artery disease undergoing major noncardiac surgery, the administration of a 3-adrenergic blocking agent throughout the period of hospitalization: 1) reduces mortality and cardiovascular events following hospital discharge; 2) is safe and well tolerated; and 3) the estimated cost savings in lives more than outweighs the cost of therapy.

### CROSS-REFERENCE

This application is a continuation of Ser. No. 09/426,792, filed Oct. 22, 1999, which is a continuation of Ser. No. 08/787,056, filed Dec. 3, 1996, which are incorporated herein by reference in its entirety and to which applications we claim priority under 35 USC §120.

### INTRODUCTION

The present invention relates to methods for reducing mortality and cardiovascular morbidity following surgery. In particular, the invention relates to the intensive postoperative administration of a pharmacologic cardiovascular agent to reduce mortality and cardiovascular complications. The invention is illustrated by way of working examples which demonstrate that in patients with, or at risk for, coronary artery disease undergoing major noncardiac surgery, the administration of a β-adrenergic blocking agent throughout the period of hospitalization: 1) reduces mortality and cardiovascular complications following hospital discharge; 2) is safe and well tolerated; and 3) the estimated cost savings in lives more than outweighs the cost of therapy.

### BACKGROUND OF THE INVENTION

Cardiovascular mortality and morbidity are prevalent and costly for the 30 million patients undergoing noncardiac surgery annually in the United States. More than one million of these patients suffer heart attacks or other cardiac complications after the operation, with about 500,000 resultant deaths during the first two postoperative years (Mangano, 1990, Anesthesiology 72: 153-184; Mangano and Goldman, 1995, N. Eng. J. Med. 333:1750-1756). In the subset of 3 million surgical patients with or at-risk for coronary artery disease, the most significant risk factors for mortality and cardiovascular morbidity are myocardial ischemia and non-fatal myocardial infarction occurring during the first week following surgery, which increases the risk of serious adverse cardiovascular outcomes by 2- to 20-fold over the two years following surgery (Mangano et al., 1990, N. Eng. J. Med. 323: 1781-1788; Mangano et al., 1992, JAMA 268:233-239; Browner et al., 1992, JAMA 268:228-232). These postoperative ischemia events appear to be related to the persistent exaggerated sympathetic response that is associated with substantial increases in heart rate throughout the in-hospital period (Rao et al, 1983 Anesthesiology 59:499-505; Gottlieb et al., 1987, J. Am. Coll. Cardiol. 10:756-760; Siliciano et al., 1990, Postoperative Myocardial Infarction: Mechanisms and Therapies, In Estafanous (ed.): Opioids in Anesthesia, Butterworth Publishers, Boston pp. 164-177; Mangano et al., 1991, J. Am. Coll. Cardiol. 17:843-850; Mangano et al., 1991, J. Am. Coll. Cardiol. 17:851-857). Studies conducted over the past decade have established the association between postoperative myocardial ischemia and post-discharge adverse outcomes, with the odds of such outcomes increasing in patients with (versus without) postoperative ischemia by 28-fold six months following surgery, 20-fold at one year, and 14-fold at two years (Mangano et al., 1990, N. Eng. J. Med. 323:1781-1788; Mangano et al., 1992, JAMA 268:233-239; Browner et al., 1992, JAMA 268:228-232; Raby et al., 1989, N. Eng. J. Med. 321:1296-1300; Slogoff and Keats, 1985, Anesthesiology 62:107-114; Eisenberg et al., 1992, JAMA 268:210-216). In addition, studies have demonstrated an association between postoperative ischemia and elevated heart rate, and have suggested that mitigation of this heart rate response may reduce the incidence and/or severity of ischemia (Rao et al. 1983, Anesthesiology 59:499-505; Gottlieb et al., 1987, J. Am. Coll. Cardiol. 10:756-760; Mangano et al., 1991, J. Am. Coll. Cardiol. 17:843-850; Mangano et al., 1991, J. Am. Coll. Cardiol. 17:851-857; Wallace et al., 1994, Anesthesiology 81:A99). In at-risk patients about to undergo major surgery, the standard practice is to control heart rate prior to surgery, to continue medication to the time of surgery, and to modulate the heart rate response during surgery using anesthetic techniques. However, following surgery heart rate is not well-controlled, increasing above preoperative levels by 30 percent or more, throughout the extended postoperative period (Mangano et al., 1992, JAMA 268:233-239; Mangano et al., 1991, J. Am. Coll. Cardiol. 17:843-850; Mangano et al., 1991, J. Am. Coll. Cardiol. 17:851-857; Raby et al., 1989, N. Eng. J. Med. 321:1296-1300; Eisenberg et al., 1992, JAMA 268:210-216). Furthermore, even brief periods of tachycardia during the postoperative period may precipitate ischemia in these patients, who also are subjected to alterations in perfusion, oxygenation and coagulation, as well as other stresses imposed by the exaggerated sympathetic and inflammatory responses to surgery. However, despite appreciation of the general problem of perioperative infarction, as well as the potentially deleterious effect of an unchecked postoperative sympathetic response, and despite recognition of the efficacy of β-blockade in ambulatory patients with coronary artery disease, clinicians have been reluctant to prescribe β-blockers following surgery, even for patients who had been maintained on β-blockers prior to admission for surgery. Such reluctance is based on several concerns, including: 1) safety—namely precipitation of postoperative heart failure, hypotension and bronchospasm; 2) efficacy—unproven for the surgical patient; and 3) cost.

Several clinical trials have investigated the effects of preoperative or intraoperative use of nitrates (Coriat et al., 1984, Anesthesiology 63: 193-196; Gallagher et al., 1986, Anesthesiology 64:785-789), β-adrenergic blockers (Stone et al., 1988, Anesthesiology 68:495-500; Magnusson et al., 1986, Br. J. Anaeth. 58:251-260; Cucchiara et al., 1986, 65-528-531), calcium channel blockers (Chung et al., 1988, Anesthesiology 69:343-347; Merin, 1987, Anesthesiology 66:111), and alpha (α)-2 agonists (Ghignone et al., 1987, Anesthesiology 67:3-10; Talke et al., 1995, Anesthesiology 82:629-633) on hemodynamics and measures of myocardial ischemia. In the studies involving β-blockers, the drugs were always administered prior to the surgical procedures. Prior to the present invention, it was not known that continuous postoperative administration of these agents would result in a reduction of cardiovascular mortality and morbidity. In particular, it was not expected that it would have any long-term beneficial effects on mortality and cardiovascular events, such as myocardial infarction, heart failure and unstable angina requiring revascularization.

### SUMMARY OF THE INVENTION

The present invention relates to methods for reducing mortality and cardiovascular morbidity following surgery by the intraoperative and postoperative administration of a therapeutic amount of a pharmacologic cardiovascular agent. In particular, it relates to the intensive postoperative administration of such an agent during hospitalization and even after hospital discharge to mitigate the sympathetic response associated with increased heart rate, increased thrombosis and increased inflammatory response, thereby reducing the incidence and/or severity of cardiovascular complications such as myocardial infarction, unstable angina, congestive heart failure, dysrhythmia, myocardial revascularization, and death.

The invention is based, in part, on the Applicant's discovery that the administration of a β-adrenergic blocker, atenolol, prior to and immediately following surgery, and continuing daily throughout the entire period of hospitalization in patients with, or at risk for, coronary artery disease undergoing noncardiac surgery, reduces mortality and serious cardiovascular complications following hospital discharge, with the early survival effects persisting for two years. Therefore, a wide variety of uses are encompassed by the present invention including, but not limited to, increasing the survival rate and decreasing cardiovascular complications in patients under surgical stress.

### BRIEF DESCRIPTION OF THE DRAWINGS

FIG. 1. Intensive postoperative administration of atenolol increases the survival of patients for two years after surgery.

FIG. 2. Intensive postoperative administration of atenolol increases cardiovascular event-free survival of patients for two years after surgery.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The present invention relates to treatment of patients undergoing surgery to reduce mortality and cardiovascular complications by the administration of a pharmacologic cardiovascular agent such as β-adrenergic blocking agent following surgery. The treatment may be continued throughout hospitalization, and even after discharge. Both the time-to-first adverse event, as well as survival and event-free survival, are significantly improved by such treatment, particularly during the first 6-8 months following surgery, with survival effects persisting for two years. In the β-adrenergic blocking agent-treated patients, survival was 90 percent at two years following surgery versus 79 percent in placebo-treated patients, and event-free survival was 83 percent versus 68 percent, respectively. Moreover, the intensive postoperative drug administration was well-tolerated in these patients, despite the prevalence of cardiac and pulmonary disease.

The invention is discussed in more detail in the subsections below, solely for the purpose of description, and not by way of limitation. Although the specific procedures and methods described herein are exemplified with the administration of atenolol immediately before and after surgery and continuing for up to seven days thereafter, they are merely illustrative for the practice of the invention. Analogous schedules, procedures, techniques and pharmacologic cardiovascular agents are equally applicable.

### I. Suitable Pharmacologic Cardiovascular Agents

The present invention relates to the intensive postoperative use of a pharmacologic cardiovascular agent to reduce mortality and morbidity following surgery. As used herein, a “pharmacologic cardiovascular agent” is an agent that mitigates cardiovascular stress responses by reducing heart rate, blood coagulation or inflammatory reactions. In a specific embodiment of the invention by way of working examples, infra, a β-adrenergic blocking agent is used to reduce heart rate. β-adrenergic receptors are expressed on different cell types, including cardiac muscle cells. These receptors are further subdivided into β₁ and β₂ receptors on the basis of their tissue distribution, both forms are coupled to a signal transducer referred to as the G protein. The binding of these receptors by a ligand results in G-protein-mediated activation of the enzyme adenylate cyclase, which causes an elevation of intracellular cyclic AMP levels as well as activities of ion channels. An increase of cyclic AMP regulates a number of downstream cellular metabolic events. Such events are manifested in an increased contraction rate of cardiac muscle cells which, in turn, promotes increased heart rate and blood pressure. Under certain circumstances of bodily stress such as surgery, these events can lead to serious cardiovascular complications, even death. In view of the foregoing observation, a number of β-adrenergic blocking agents or antagonists have been tested clinically for the treatment of hypertension, ischemic heart disease and certain cardiac arrhythmias.

The interactions between hormones such as epinephrine and β-adrenergic receptors have been well studied in the art. The binding of epinephrine to β-adrenergic receptors activates adenylate cyclase, and a number of measurable downstream cellular events. Since the β-adrenergic receptor has been molecularly cloned and expressed in receptor-negative cells, the ability of such cells to activate adenylate cyclase in response to epinephrine may be conveniently tested in an in vitro assay system. Alternatively, a β-adrenergic receptor-positive cell line may also be used. For the purpose of this invention, any substance that blocks or interferes with the activation of adenylate cyclase by a ligand such as epinephrine upon its binding to the β-adrenergic receptors is a β-adrenergic blocking agent suitable for use in the present invention.

In accordance with the methods of the invention, β-adrenergic blocking agents encompass both β₁-selective and non-selective blockers. However, β₁-selective blockers are preferred because they exert minimal effects on the β-adrenergic receptors on non-cardiac muscle cells. Examples of suitable blocking agents include, but are not limited to, atenolol, metaprolol, esmolol, acebutolol, practolol, alprenolol, propanolol, nadolol, timolol, pindolol, labetalol, sotalol and oxprenolol. The aforementioned agents are commercially available or may be readily prepared by methods well known in the art (Goodman and Gilman's, The Pharmacological Basis of Therapeutics, 1990, eighth ed., Pergamon Press).

Additionally, other pharmacologic agents with known cardiovascular effects in reducing heart rate, blood coagulation and inflammation are also suitable for use in the present invention, and such agents include, but are not limited to, α-2 agonists such as clonidine, anti-ischemic agents which encompass calcium channel blockers such as verapamil and nifedipine, angiotensin converting enzyme (ACE) inhibitors such as lisinopril and enalapril, and nitrates (nitroglycerin), antiplatelet agents such as aspirin and dipyridamole, antithrombotics such as coumadin, heparin and streptokinase, and the like (Physicians' Desk Reference, 1996, 50th Edition, Medical Economics).

### II. Dosage and Formulation

The agents described in Section 5.1, supra, may be administered into a patient for the reduction of mortality and cardiovascular morbidity following surgery by any means that produces contact of the active agent with the agent's site of action in the body of the patient. They can be administered by any conventional means available for use in conjunction with Pharmaceuticals, either as individual therapeutic agents or in a combination of therapeutic agents. Each can be administered alone but is generally administered with a pharmaceutical carrier selected on the basis of the chosen route of administration and standard pharmaceutical practice. The pharmaceutical compositions of the invention may be adapted for oral, parenteral or topical administration, and may be in unit dosage form, in a manner well known to those skilled in the pharmaceutical art. Parenteral administration includes, but is not limited to, injection subcutaneously, intravenously, intraperitoneally or intramuscularly.

In accordance with this aspect of the invention, a pharmacologic cardiovascular agent is administered during and after the surgical operation, and if tolerated by the patient, it may be continued for 3-7 days until the patient is discharged from the hospital, and following hospital discharge. The administration may begin immediately after surgery and continuing daily through hospital discharge and following hospital discharge. Alternatively, the administration may be initiated only after the first clinical manifestation of cardiovascular stress such as high blood pressure, hypertension, myocardial ischemia or infarction, increased heart rate relative to the preoperative rate, clotting abnormalities and inflammatory reaction such as a rise in body temperature, or a local tissue reaction involving infiltration of white blood cells or other inflammatory mediators. In addition, the administration may begin prior to surgery, with the preferred timing of administration being from 1 day to 1 hour before surgery. The dose administered will, of course, vary depending upon known factors, such as: the pharmacodynamic characteristics of the particular agent and its mode and route of administration; the age, health, height and weight of the recipient; the nature and extent of the symptoms; the kind of concurrent treatment(s); the frequency of treatment(s); and the effect desired. A daily dose of active ingredient can be expected to be about 0.1-500 mg per patient, with the preferred dose being 5-100 mg given in two separate doses.

More specifically, atenolol may be used orally at 50-100 mg/day and at 5-10 mg BID intravenously. Labetalol may be used orally at 200-400 mg BID and at 20 mg bolus intravenously over 2 minutes with a repeated dose of 40-80 mg over 10 minutes up to a maximum dose of 300 mg. Clonidine may be used orally at 0.2-1.2 mg/day and at 0.1-0.3 mg by skin patch every 7 days. Nitroglycerine may be used orally at gr 1/100-gr 1/400 sublingual with a repeated dose for 15-30 minutes and at 1-10/μg/kg/minute. Veraparmil may be used orally at 240-320 mg/day and at 0.075-0.15 mg/kg intravenously over 2 minutes. Nifedipine may be used orally at 10-20 mg TID. Lisinopril may be used orally at 20-40 mg/day. Enalapril may be used orally at 10-40 mg/day and at 1.25 mg intravenously for 6 hours. Aspirin may be used orally at 325-650 mg/day or BID. Dipyridamole may be used orally at 50-400 mg/day and at 0.142 mg/kg/minute intravenously over 4 minutes up to a total dose of 0.5 mg/kg. Coumadin may be used orally at 10-15 mg/day for 3 days followed by 2-3 mg/day. Heparin may be used at 1000-5000U intravenous push or 5000-7500U intravenous bolus followed by adjustment according to PTT. Streptokinase may be used intravenously at 20,000 IU bolus followed by a dose of 2,000 IU/minute for 60 minutes.

Dosage forms (compositions suitable for administration) contain from about 1 mg to about 500 mg of active ingredient per unit. In these pharmaceutical compositions, the active ingredient is ordinarily present in an amount of about 0.5-95% by weight based on the total weight of the composition.

The active ingredient can be administered orally in solid or semi-solid dosage forms, such as hard or soft-gelatin capsules, tablets, or powders, or in liquid dosage forms, such as elixirs, syrups, or suspensions. It can also be administered parenterally, in sterile liquid dosage forms. Other dosage forms are potentially possible such as patches or ointment or transdermal administration.

Gelatin capsules or liquid-filled soft gelatin capsules may contain the active ingredient and powdered or liquid carriers, such as lactose, lecithin starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar-coated or film-coated to mask any unpleasant taste and to protect the tablet from the atmosphere, or enteric-coated for selective disintegration in the gastrointestinal tract. Liquid dosage forms for oral administration can contain coloring and/or flavoring to increase patient acceptance.

In general, water, oil, saline, aqueous dextrose (glucose), polysorbate and related sugar solutions and glycols such as propylene glycol or polyethylene glycols are suitable carriers for parenteral solutions. Solutions or emulsions for parenteral administration preferably contain about 5-15% polysorbate 80 or lecithin, suitable stabilizing agents, and if necessary, buffer substances. Antioxidizing agents including, but not limited to, sodium bisulfite, sodium sulfite, or ascorbic acid, either alone or combined, are suitable stabilizing agents. Also used are citric acid and its salts and sodium EDTA. In addition, parenteral solutions can contain preservatives, including but not limited to, benzalkonium chloride, methyl- or propyl-paraben, and chlorobutanol.

Suitable pharmaceutical carriers are further described in Remington's Pharmaceutical Sciences, 1990, 17th ed., Mack Publishing Company, Easton, Pa., a standard reference text in this field, which is incorporated herein by reference in its entirety.

### III. Methods of Using Pharmacologic Cardiovascular Agents in Patients Undergoing Surgery

The methods of the invention are generally applicable to patients undergoing surgery to reduce their long-term mortality and cardiovascular morbidity. The methods are particularly useful for patients who have or are at risk for coronary artery disease. The cardiac risk factors include hypertension, smoking, diabetes mellitus, age over 65 and cholesterol level>6.2 mmol/liter.

The methods of the invention are applicable to patients undergoing any form of surgery that causes cardiovascular stress whether or not it is cardiac surgery. The types of surgery include, but are not limited to, intraabdominal, orthopedic, neurological, intrathoracic, head and neck, vascular and general surgery.

Intensive administration of the pharmacologic cardiovascular agents may begin immediately after surgery. However, in the case of coronary bypass, it may begin intraoperatively, either prior to or following institution of cardiopulmonary bypass or at any time during postoperative hospitalization. Alternatively, initiation of therapy may await the first manifestations of cardiovascular stress. Markers for such stress include high blood pressure, hypertension, myocardial infarction, unstable angina, tachycardia, clotting abnormalities and inflammatory response. Intensive therapy refers to daily administration of the pharmacologic agents until reduction of symptoms of cardiovascular stress or hospital discharge.

### IV. Example: Administration of a Beta-Blocker Reduces Mortality and Cardiovascular Morbidity Following Surgery

### Materials and Methods

1. Patient Population

Eligible patients included those with, or at risk for coronary artery disease and scheduled for elective noncardiac surgery requiring general anesthesia at the San Francisco Veterans Affairs Medical Center. The specific inclusion and exclusion criteria have been described previously (Mangano et al., 1990, N. Eng. J. Med. 323:1781-1788; Mangano et al., 1992, JAMA 268:233-239). A maximum of one patient per day was enrolled and, of the 204 patients consenting to the study, 200 were enrolled randomized and received study drug.

2. Beta-Adrenergic Blocking Agent

Patients were randomized to receive either atenolol “TENORMIN” (Zeneca Pharmaceuticals) or placebo prior to induction of anesthesia, immediately following surgery, and daily throughout their hospitalization (up to 7 days). Drug assignment, study physicians, treating clinicians, and data analysis personnel were blinded to study group throughout all phases of this trial. Intravenous and oral preparations of active drug atenolol and placebo were prepared by the hospital pharmacy with a computer-generated randomized list retained only by the pharmacy and maintained confidential until formal study unblinding following database closure.

Intravenous preparation consisted of two-10-ml syringes, each containing 5 mg atenolol or placebo; oral preparation consisted of two 50 mg tablets of atenolol, or two placebo tablets. Approximately one hour prior to surgery, patients entered the preoperative area and blood pressure was recorded with an automated cuff and 5-lead continuous electrocardiograph. Thirty-minutes prior to entry into the operating room, intravenous administration of study drug began. Exclusion criteria for study drug administration were heart rate <55 bpm, systolic blood pressure <100 mm Hg, or evidence of congestive heart failure, third degree heart block, or bronchospasm (ISIS-I protocol, 1986, Lancet 2:56-66). If none of these criteria was present, the first syringe of study drug was infused over five minutes, the patient was observed for an additional five minutes, and, if no exclusion criteria developed, the second syringe was infused over five minutes. Immediately following surgery, the study drug was again given using the identical technique applied prior to surgery. On the morning of the first postoperative day, and daily thereafter until the patient was discharged from the hospital (up to a maximum of seven days), patients received study drug every 12 hours using the same technique for intravenous infusion, or orally (if able) at which time a tablet of atenolol (0, 50 or 100 mg) or placebo was given daily. If heart rate was >65 bpm and systolic blood pressure >100 mm Hg, 100 mg atenolol (or placebo) was given orally; if heart rate was >55 but <65 bpm and systolic blood pressure >100 mm Hg, 50 mg atenolol (or placebo) was administered; if heart rate was <55 bpm or systolic blood pressure <100 mm Hg, 0 mg atenolol (or placebo) was given. No treating clinician was allowed to observe study drug administration either prior to, or after, surgery.

3. Clinical Care

All patients received general anesthesia with endotracheal intubation; preoperative medications were continued until the time of surgery, with beta-blockers replaced by study drug on the morning of surgery. There were no other protocol-based restrictions of anesthetic or surgical technique, and clinical decisions were not controlled by study protocol. Perioperative information was recorded and analyzed for possible confounding effects, and included: type and duration of surgery, anesthetic techniques, fluid and blood loss and replacement, cardiovascular medications, hemodynamics, electrocardiographic data, and adverse events.

4. Clinical Follow Up and Outcome Measurements

Of the 200 patients enrolled, 194 were discharged following surgery and six patents died during hospitalization—three cardiac deaths secondary to myocardial infarction (two placebo and one atenolol), and three noncardiac deaths, with two secondary to metastatic cancer (both atenolol), and one with pulmonary failure secondary to massive infusion for fluid loss (atenolol). Of the 194 patients discharged, outcome data were collected in 192 patients (99%), with two patients (one placebo and one atenolol) lost to followup. At six months, one year and two years following surgery, study physicians conducted scheduled research study visits that were independent of usual clinical care. Data collected during each visit included history and physical examination, a 12-lead electrocardiogram, and review of all medical records, medications and hospital admissions. Cardiac death was diagnosed if the patient died of either a myocardial infarction, dysrhythmia or congestive heart failure caused primarily by a cardiac condition. Myocardial infarction required the following: 1) development of new Q waves (as defined by Minnesota Code 1-1-1-21-2-7); or 2) new persistent ST-T wave changes (Minnesota Code 4-1 or 4-2; 5-1 or 5-2) associated at the time of hospitalization with elevation of total creatinine kinase and CK-MB isoenzyme; or 3) necropsy evidence of acute myocardial infarction; or 4) hospital record documentation of myocardial infarction (Mangano et al., 1990, N. Eng. J. Med. 323:1781-1788). Unstable angina required severe precordial chest pain that lasted at least 30 minutes, was unresponsive to standard therapeutic maneuvers and associated with transient ST-segment or T-wave changes without development of Q waves or diagnostic enzyme abnormalities. The diagnosis of congestive heart failure required symptoms or signs of pulmonary congestion (shortness of breath and rales), signs of new left or right ventricular failure (cardiomegaly, S3, jugular venous distention, and peripheral edema), abnormal results on chest radiography (vascular redistribution, interstitial edema, and alveolar 5 edema), and a change in medication involving (at least) treatment with diuretic agents (Mangano et al., 1990, N. Eng. J. Med. 323:1781-1788).

Outcomes were prescribed by study protocol, and the primary outcome was all-cause mortality during the two years following hospital discharge. The secondary outcome was combined consisting of: 1) myocardial infarction; or 2) unstable angina or congestive heart failure requiring hospital admission and clinical diagnosis and treatment, or 3) myocardial revascularization (coronary artery bypass graft surgery or percutaneous transluminal angioplasty), or 4) death. Autopsy data, if available for patients who died over 5 the two-year period, were reviewed centrally at the core laboratory (Ischemia Research and Education Foundation) by a pathologist blinded to patient treatment group.

5. Statistical Analysis

The study was designed to allow assessment of tolerance, in-hospital events (hemodynamic changes, dysrhythmia, ischemia), and adverse cardiovascular outcomes occurring over the two years following surgery. A sample size of 200 patients was calculated based on the following assumptions: 1) duration of enrollment and followup=0.48 months; 2) two-year mortality, cardiovascular morbidity and in-hospital event rates=0.23, 0.28 and 0.41, respectively (Mangano et al., 1990, N. Eng. J. Med. 323:1781-1788; Mangano et al., 1992, JAMA 268:233-239; Browner et al., 1992, JAMA 268:228-232); 3) followup rate=0.96 (Mangano et al., 1992, JAMA 268:233-239; Browner et al. 1992, JAMA 268:228-232); 4) α=0.05, β=0.2, effect size=0.5; and 5) the alternative safety and efficacy hypotheses are two-tailed and one-tailed, respectively (Mangano, 1990, Anesthesiology 72:153-184; Stone et al., 1988, Anesthesiology 68:495-500; Magnusson et al., 1986, Br. J. Anaesth. 58:251-260; Cucchiara et al., 1986, Anesthesiology 65:528-531; Wallace et al., 1994, Anesthesiology 81:A99). Using the log-rank survival test for sample size estimation (BMDP statistical Software Inc., 1992, SOLO Power Analysis), it was calculated that 198 patients would be necessary for mortality assessment and 158 patients for combined outcome, and, using Z statistic, 170 patients for in-hospital event assessments. Mortality risk in different categories (all-cause mortality, cardiac mortality, noncardiac mortality, at 6 months, 1 year, and 2 years) was compared using Kaplan-Meier methods, as was event-free survival after discharge. Univariable predictors of two-year mortality were identified using the Cox proportional hazards regression techniques (SAS Institute Inc., 1992, Release 6.07:345-379) after first verifying that assumption of the hazards model was valid (Kaplan and Meier, 1958, J. Am. Stat. Assoc. 53: 457-481; SAS Institute, Inc., 1985, Statistical Analysis System, SAS User's Guide). Predictors with P<0.10 were entered into multivariable models and a series of models was constructed by adding variables, as long as the resulting multivariable model had a lower Chi-square P value than competing models. Analyses were performed using Statistical Analysis System Software (SAS Institute, Inc., Gary, N.C.).

### Results

Study patients were middle-aged or elderly who smoked and had a history of hypertension and chronic medical problems. There was no difference between groups, except that the atenolol group had a higher incidence of treatment for hypertension.

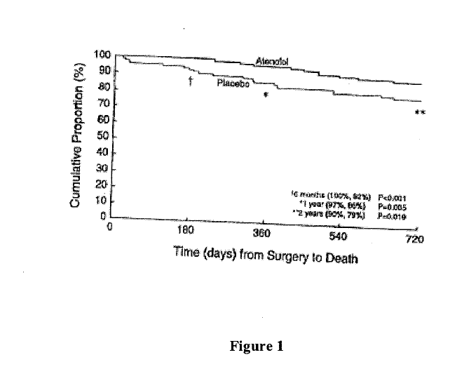

Thirty patients (15.6 percent) died over the two-year outcome period (Table 1). Twenty-one of these deaths (12 cardiac-related) occurred in the placebo group versus 9 deaths (4 cardiac-related) in the atenolol group, representing a 57 percent reduction by atenolol in all-cause mortality (P=0.019), and a 67 percent reduction in cardiac mortality (P=0.033). The principal effect of atenolol therapy was on cardiac-related outcomes occurring over the first 6-8 months (one noncardiac death versus 10 deaths with 7 cardiac-related; P<0.001), with the time to first death being 19 days in the placebo group versus 237 days in the atenolol group. Thereafter there was no substantial effect; however, the early difference in survival between groups was preserved at one year A versus 14 deaths; P=0.005) and two years (9 versus 21 deaths; P=0.019), with survival 3 significantly increased over all time periods in the atenolol group (FIG. 1).

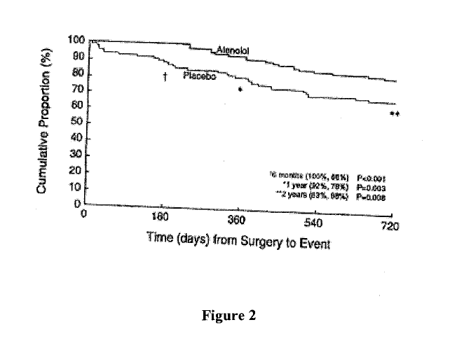

Atenolol-treated patients had a significant decrease in the rate of cardiac events within six months following surgery (0 atenolol patients versus 12 placebo patients; P<0.001), a 3-fold decrease within one year (7 atenolol patients versus 22 placebo patients; P=0.003), and a 2-fold decrease within two years following surgery (16 atenolol patients versus 32 placebo patients; P=0.008). The principal effect occurred over the first 6 to 8 months, with the time-to-first adverse event being 6 days in the placebo group versus 158 days in the atenolol group. Thereafter, 0 there was no substantial effect; however, the early difference in event-free survival was preserved over the two years following surgery (FIG. 2).

During treatment, the average heart rate was significantly lower in the atenolol group (75 bpm versus 87 bpm; P<0.001), as was the maximum heart rate (113 bpm versus 5 130 bpm; P<0.001). Multivariable correlates associated with survival at two years are listed in Table 2, and demonstrate association between survival and a history of diabetes mellitus and atenolol therapy, with atenolol improving survival in diabetics at two years by approximately 75 percent (hazard ratio, 0.25; P=0.03). Similarly, in atenolol-treated patients, the presence of diabetes was not associated with increased risk of mortality (hazard ratio, blockers, calcium channel blockers or nitrates, either prior to hospital admission or following hospital discharge. More than 85 percent of patients tolerated intravenous atenolol administration prior to and immediately following surgery, and oral administration during the postoperative period, with more than 60 percent tolerating the full dose of atenolol (10 mg intravenously or 100 mg orally) (Table 3). In approximately 10% of patients, intravenous administration of atenolol prior to or after surgery was associated with 20% or more decrease in systolic blood pressure or heart rate (Table 3); however, no patient developed systolic blood pressure <90 mm Hg or heart rate <40 bpm, or required 5 therapy. Oral administration was not associated with an increased incidence of hypotension or bradycardia, or other events. The treatment effect found in this trial cannot be attributed to inhomogeneity between groups at baseline; in fact, a larger proportion of the atenolol-treated patients 0 had cardiovascular disease prior to surgery, and had a greater number of risk factors known to affect cardiovascular complications following surgery (Mangano et al., 1990, N. Eng. J. Med. 323:1781-1788; Goldman et al., 1977, N. Eng. J. Med. 297:845-850; Detsky et al., 1986; Arch. Intern. Med. 146:2131-2134; Hollenberg et al., 1992, JAMA 268:205-209). The results also cannot be explained by differences in surgical technique, hospitalization, or preoperative, postoperative or discharge cardiovascular medication use, specifically 3-blockers and calcium channel blockers. A substantial portion of all variables was distributed evenly, and the variables which may not have been, such as treatments for heart failure or diabetes, were shown not to affect the conclusions of this trial.

The patient population represents approximately 10 percent of the 30 million patients undergoing noncardiac surgery (or 3 million patients), and even assuming an atenolol effect of one-fifth of the 57 percent effect found in the clinical trial disclosed herein (or 11 percent), the 5 intensive postoperative administration of a β-blocker may save 33,000 lives per year at a cost of less than 100 dollars per patient (conservative estimate for one week of atenolol therapy) for the 3 million at-risk patients, or an overall cost equalling 9,000 dollars per live saved.

The present invention is not to be limited in scope by the exemplified embodiments, which are intended as illustrations of individual aspects of the invention. Indeed, various modifications for the invention in addition to those shown and described herein will become apparent to those skilled in the art from the foregoing description and accompanying drawings. Such modifications are intended to fall within the scope of the appended claims.

All publications cited herein are incorporated by reference in their entirety.

TABLE 1
Long-term Deaths

| | | | | | TIME TO | |
|---|---|---|---|---|---|---|
| | PATIENT | | | | DEATH | |
| GROUP | NO. | AGE | CV RISK FACTORS | TYPE OF SURGERY | (DAYS) | CAUSE OF DEATH |
| Placebo | 1 | 65 | DM, HTN, PVD, age ≧65 | Peripheral Vascular | 19 | Massive GI hemorrhage |
| | 2 | 67 | HTN, age ≧65 | Peripheral Major | 24 | Sudden Cardiac death |
| | 3 | 77 | PVD, age ≧65 | AAA Repair | 33 | CHF, severe CAD |
| | 4 | 64 | DM, SM | Peripheral Major | 35 | CHF |
| | 5 | 67 | CAD, DM, HTN, PVD, age ≧65 | Peripheral Vascular | 97 | Cardiac Arrest |
| | 6 | 65 | SM, PVD, age ≧65 | AFBG | 112 | Acute bronchopneumonia, COPD |
| | 7 | 75 | DM, PVD, age ≧65 | Carotid | 162 | Sudden cardiac death |
| | 8 | 69 | HTN, age ≧65 | Intra-abdominal | 185 | Adeno CA, colon |
| | 9 | 78 | HTN, PVD, age ≧65 | Carotid | 197 | Acute MI, post PTCA |
| | 10 | 62 | CAD, HTN, PVD, SM | Peripheral Vascular | 236 | Acute MI |
| | 11 | 80 | CAD, HTN, age ≧65 | Intra Abdominal | 303 | Cardiac arrest |
| | 12 | 64 | CAD, SM, PVD | Peripheral Vascular | 325 | Sepsis |
| | 13 | 69 | CAD, DM, HTN, age ≧65 | Intra-abdominal | 328 | Small bowel obstruction 2° to |
| | | | | | | CA, prostate |
| | 14 | 77 | CAD, DM, HTN, SM, PVD, age ≧65 | Intra-abdominal | 376 | Acute MI |
| | 15 | 65 | DM, SM, age ≧65 | Peripheral Major | 384 | Bladder CA |
| | 16 | 65 | CAD, DM, age ≧65 | Intra-abdominal | 517 | Sepsis 2° bowel obstruction |
| | 17 | 75 | CAD, DM, PVD, age ≧65 | Peripheral Vascular | 517 | Cardiac Arrest |
| | 18 | 75 | DM, age ≧65 | Intra-abdominal | 629 | Metastatic CA, colon |
| | 19 | 81 | DM, age ≧65 | Peripheral Major | 658 | Acute MI, ARDS |
| | 20 | 69 | DM, HTN, age ≧65 | Intra-abdominal | 734 | Post MI CVA |
| | 21 | 66 | HTN, SM, PVD, age ≧65 | Carotid | 755 | Periotonitis 2° to perforation of ileum |
| Atenolol | 1 | 56 | SM, PVD | Peripheral Vascular | 237 | Respiratory failure |
| | 2 | 56 | CAD, PVD, HTN, SM | Peripheral Vascular | 295 | Ventricular tachycardia |
| | 3 | 78 | CAD, DM, HTN, age ≧65 | Intra-abdominal | 327 | Severe CAD, sepsis |
| | 4 | 66 | CAD, HTN, SM, age ≧65 | Peripheral Major | 385 | Sepsis, ALS |
| | 5 | 67 | DM, THN, age ≧65 | Peripheral Major | 416 | Metastatic renal CA |
| | 6 | 74 | CAD, DM, HTN, age ≧65 | Peripheral Minor | 481 | CHF post CABG |
| | 7 | 79 | HTN, age ≧65 | Intra-abdominal | 529 | ARDS |
| | 8 | 70 | CAD, HTN, PVD, age ≧65 | Carotid | 582 | Cardiac arrest, lung CA |
| | 9 | 78 | HTN, SM, PVD, age ≧65 | Carotid | 656 | Metastatic squamous cell CA, larynx, lung |

CAD denotes coronary artery disease (consisting of previous CABG, MI, typical angina, chest pain with ischemic ECG responsive to exercize, scintigraphic evidence of myocardial perfusion defect, or abnormal coronary angiography), CHF congestive heart failure, COFD chronic obstructive pulmonary disease, ARDS adult respiratory distress syndrome, ALS amyotrophic lateral sclerosis, AFBF aorto-femoral bypass graft, AAA abdominal aortic aneurysm, CA cancer, DM diabetes mellitus, HTN hypertension, PVD vascular disease, and SM smoking.

TABLE 2
Variables Associated with 30 Deaths among
200 Patients Undergoing Non-cardiac Surgery

| | HAZARDS | CONFIDENCE | |
|---|---|---|---|
| PREDICTOR | RATIO | INTERVAL | P VALUE |
| Univariable models | | | |
| Atenolol | 0.4 | 0.2-0.9 | 0.03 |
| Diabetes mellitus | 3.1 | 1.4-6.8 | 0.01 |
| Oral Hypoglycemic treatment | 2.6 | 1.1-6.2 | 0.03 |
| Insulin Treatment | 2.6 | 1.0-6.9 | 0.05 |
| Holter ischcmia postop | 2.3 | 1.0-5.3 | 0.04 |
| days 0-2 | | | |
| Multivariate models | | | |
| Diabetes mellitus | 2.8 | 1.4-6.2 | |
| Atenolol | 0.5 | 0.2-1.1 | |

TABLE 3
Use of Cardiovascular Medication

| | PERCENT OF SUBJECTS | | | | | | | | | | | |
|---|---|---|---|---|---|---|---|---|---|---|---|---|
| | | | | CALCIUM | | | | | | ACE | | |
| | β-BLOCKER | | | CHANNEL BLOCKER | | | NITRATE | | | INHIBITOR | | |
| | ATEN- | PLA- | | ATEN- | PLA- | | ATEN- | PLA- | | ATEN- | PLA- | |
| | OLOL | CEBO | P-VALUE | OLOL | CEBO | P-VALUE | OLOL | CEBO | P-VALUE | OLOL | CEBO | P-VALUE |
| Prior to | 18 | 8 | 0.02¹ | 22 | 34 | 0.11³ | 8 | 13 | 0.36 | 22 | 8 | 0.003⁷ |
| Admission | | | | | | | | | | | | |
| At hospital | 13 | 7 | 0.12² | 18 | 27 | 0.18⁴ | 7 | 15 | 0.09⁶ | 19 | 6 | 0.003⁸ |
| discharge | | | | | | | | | | | | |
| At 6 months | 14 | 8 | 0.27 | 19 | 30 | 0.10⁵ | 16 | 22 | 0.30 | 15 | 18 | 0.58 |
| At 12 months | 17 | 14 | 0.61 | 24 | 30 | 0.36 | 19 | 27 | 0.25 | 24 | 24 | 0.98 |
| At 24 months | 16 | 14 | 0.79 | 19 | 25 | 0.36 | 14 | 18 | 0.51 | 18 | 22 | 0.61 |

*-Chi-square statistics were used to test the difference between the two treatment groups.
†-Number of atenolol patients = 99; number of placebo patients-101.
§-Number of atenolol patients = 94; number of placebo patients-99.
¶-Number of atenolol patients 93; number of placebo patients-91.
∥-Number of atenolol patients = 93; number of placebo patients-91.
¥-Number of atenolol patients = 90; number of placebo patients-85.
¹Odds ratio for pre-admission β-blocker use and 2-year mortality = 0.80 (P = 0.73)
²Odds ratio for discharge β-blocker use and 2-year mortality = 0.61 (P − 0.52)
³Odds ratio for pre-admission calcium blocker use and 2-year mortality = 1.06 (P − 0.90)
⁴Odds ratio for discharge calcium blocker use and 2-year mortality = 0.85 (P = 0.74)
⁵Odds ratio for 6-month calcium blocker use and 2-year mortality = 1.05 (P = 0.92)
⁶Odds ratio for discharge nitrate use and 2-year mortality = 1.32 (P − 0.64)
⁷Odds ratio for pre-admission ACE inhibitor use and 2-year mortality = 1.45 (P = 0.50)
⁸Odds ratio for discharge ACE inhibitor use and 2-year mortality = 1.17 (P = 0.79)

What is claimed is:

1. A method for reducing cardiovascular disease complications in a patient following surgery comprising the sequential steps of:
(i) intravenously administering to the patient a β₁-adrenergic blocking agent immediately after surgery; and thereafter
(ii) administering the agent daily thereafter until symptoms of cardiovascular stress are reduced,
wherein the agent is administered near the maximum effective dose of the agent while the patient's heart rate is greater than or equal to 65 bpm, the patient's systolic blood pressure is greater than or equal to 100 mm Hg, and the patient evidences no congestive heart failure, third degree heart block, or bronchospasm.

2. The method of claim 1 in which the agent is administered in step (ii) daily in the period after surgery until hospital discharge.

3. The method of claim 2 in which the agent is administered in step (ii) daily in the period after surgery for at least three days.

4. The method of claim 2 in which the agent is administered in step (ii) daily in the period after surgery for up to seven days.

5. The method of claim 1 in which the f₁-adrenergic blocking agent is atenolol.

6. The method of claim 1 in which the patient suffers from coronary artery disease.

7. The method of claim 1 wherein the patient is at risk for coronary artery disease.

8. The method of claim 1 in which the patient has had previous vascular surgery or has at least two of the following cardiac risk factors: older than 65 years, hypertensive, current smoker, serum cholesterol level of at least 6.2 mmol/L, or diabetes mellitus.

9. The method of claim 1 in which the agent is atenolol and the maximum effective dose is about 100 mg/day orally or about 10 mg BID intravenously.

10. The method of claim 1 wherein the agent is administered in step (ii) daily for six months following surgery.

11. The method of claim 1 wherein the agent is administered in step (ii) daily for eight months following surgery.
